(19) United States
(12) Patent Application Publication
(10) Pub. No.: US 2015/0351719 A1
(43) Pub. Date: Dec. 10, 2015

(54) ULTRASONIC IMAGING DEVICE AND CART FOR ULTRASONIC IMAGING DEVICE

(71) Applicant: HITACHI ALOKA MEDICAL, LTD., Mitaka-shi, Tokyo (JP)

(72) Inventors: Atsushi NINOMIYA, Tokyo (JP); Katsumi USAMI, Tokyo (JP); Masaru YOKOYAMA, Tokyo (JP); Kazuyuki YANASE, Tokyo (JP)

(21) Appl. No.: 14/655,458

(22) PCT Filed: Dec. 11, 2013
(86) PCT No.: PCT/JP2013/083197

§ 371 Date: Jun. 25, 2015

(30) Foreign Application Priority Data
Dec. 28, 2012 (JP) 2012-286564

(51) Int. Cl. A61B 8/00; B62B 3/10

(52) U.S. Cl. CPC A61B 8/4405; B62B 3/10

(57) ABSTRACT

An ultrasonic imaging device has a cable that is prevented from being dragged on a floor surface during examination and when a probe is not in use. The probe slides in and out smoothly and the cable can be pulled out without excessive force during examination. The ultrasonic imaging device includes a cart portion mounted with an instrument body, a table supported by the cart portion and configured to mount the instrument body, a support mechanism for the table, and a housing to house the support mechanism. A probe holder is provided on a side of the table. A base seat of the cart portion includes a raised portion surrounding an outer periphery of the housing fixed to the base seat, and a storage recess is provided to store a probe cable and is formed between the raised portion of the base and the outer periphery of the housing.

### TECHNICAL FIELD

The present invention relates to a mobile ultrasonic imaging device, particularly, an ultrasonic imaging device in which operability at the time of storing and putting in/out a probe is improved.

### BACKGROUND ART

An ultrasonic imaging device is widely utilized as a relatively simple and non-invasive medical ultrasonograph, and a mobile ultrasonic imaging device which can be used not only in an examination room but also in a ward of a hospital and outside the hospital is widely used. In the ultrasonic imaging device, an ultrasonic probe called a probe is placed on an examination target, an ultrasonic signal is transmitted to the inside of a body, and an echo reflected in the inside is detected by the probe. Various kinds of probes having different shapes and functions are prepared as the probes. A probe suitable for an examination site and an examination purpose is selected at the time of examination, and connected to a device for use.

The mobile ultrasonic imaging device is provided with a probe holder capable of storing a plurality of probes such that the plurality of probes can be used as needed. Many kinds of probe holders having various kinds of shapes and forms have been proposed, but in most cases, the probe holder is mounted on a table where an operation panel is provided or beside a table where an operation panel is set (PTL 1 and PTL 2).

The probe includes a connector to be connected to a probe connector on the ultrasonic imaging device side, and the connector is connected, via a cable, to a body provided with an array of ultrasonic transducers. The cable is needed to have a certain length because it is necessary to pull around the probe from a place where the ultrasonic imaging device is installed to an examination site of a patient. However, in the case of not using the probe, there may be problems in which a long cable is obstructive at the time of examination and at the time of moving the ultrasonic imaging device, and the cable itself is stained contacting a floor surface or entangled with other probe cables.

In contrast, in the technology disclosed in PTL 1, a technology in which a probe holder provided with a slit is rotatably mounted on a probe holder holding unit is proposed. According to this technology, a cable is held at the probe holder without coming off from the slit by rotating the probe holder so as to close the slit at the time of examination, thereby preventing the cable from contacting the floor surface. Further, PTL 2 discloses a technology in which a housing-like cable storage unit is integrally formed immediately below a probe holder portion, and a roller is provided at the storage unit so as to pull apart of the cable exposed outside the storage unit into the storage unit.

### CITATION LIST

### Patent Literature

PTL 1: JP 2000-333949 A

PTL 2: JP 2011-36302 A

### SUMMARY OF INVENTION

### Technical Problem

According the technology disclosed in PTL 1, the cable exposed outside the connector is pulled around via the probe holder portion, and therefore, an entire length of the cable cannot be utilized. In other words, a freely usable length of the cable is to be not a length from the connector to the probe body but a length from the probe holder portion to a probe body. Consequently, an examinable range is reduced at a place where the device is installed.

According to the technology disclosed in PTL 2, in the case where a plurality of probe cables is entangled inside the storage unit, the cable may be damaged in the case of pulling the cable in such a state. Further, since the cable pulled out from the storage unit for examination is bent at a nearly a right angle in an opening portion of the storage unit, there may a problem in which the cable can be neither smoothly pulled out nor pulled around.

Further, some mobile ultrasonic imaging devices are provided with functions to rotate a table mounted with an operation panel and a display panel, and adjust a position in a vertical direction. In the case of applying, to the technology disclosed in PTL 2, the ultrasonic imaging device provided with such a movable table, the probe holder fixed to the movable table and the cable storage unit are moved integrally. However, in the case of moving the cable storage unit having relatively large capacity, some inconvenience may be caused in work in a peripheral area and operability by moving the movable table may be deteriorated.

The present invention provides an ultrasonic imaging device in which the cable is prevented from being dragged on a floor surface at the time of both examination and not using a probe, and further the cable can be smoothly pulled around without excessive force applied to the cable during examination.

### Solution to Problem

To solve the above-described problems, the present invention is characterized in that a probe cable storage recess which continues from a side surface to a front surface is provided between a base of an ultrasonic imaging device including the base and a housing fixed to the base. More specifically, an ultrasonic imaging device according to the present invention includes:

an instrument body including a display unit and an operation unit;

a cart portion supporting the instrument body;

a top plate portion supported by the cart portion and mounted with the operation unit; and

a probe to be connected to the instrument body via a cable,

wherein the top plate portion includes a probe holder portion located adjacent to a surface mounted with the operation unit and configured to hold the probe, and the cart portion is formed with a storage recess continuous to a side surface from a front surface where an operator faces at the time of ultrasonic examination.

### Advantageous Effects of Invention

Since the probe cable storage recess continuously formed from the side surface to the front surface is provided, not only the cable is prevented from contacting a floor surface, but also the cable can be smoothly pulled around without resistance regardless of a pulling direction at the time of pulling out the cable from the storage recess.

### BRIEF DESCRIPTION OF DRAWINGS

FIG. 1 is a perspective view illustrating an entire ultrasonic imaging device according to an embodiment of the present invention.

FIG. 2 is a front view illustrating the ultrasonic imaging device in FIG. 1.

FIG. 3 is a plan view illustrating the ultrasonic imaging device in FIG. 1.

FIG. 4 is a side view illustrating the ultrasonic imaging device in FIG. 1.

FIG. 5 is a functional block diagram of an instrument body.

FIG. 6 is a diagram illustrating an exemplary probe.

FIG. 7 is a plan view of a probe holder portion.

FIG. 8 is a side view illustrating the probe holder portion together with a side section of a cup.

FIG. 9 is a front view of the probe holder portion.

FIG. 10 is a perspective view illustrating a probe cup.

FIG. 11 is a plan view illustrating a cart portion of the ultrasonic imaging device in FIG. 1.

FIG. 12(a) is a diagram schematically illustrating a side surface of the cart portion in FIG. 11, and FIG. 12(b) is a partial cross-sectional view of a storage recess.

FIG. 13 is an explanatory diagram to describe a support mechanism for a top plate portion.

FIG. 14 is an explanatory diagram to describe the support mechanism for the top plate portion.

FIG. 15 is an explanatory plan view to describe pivot of the top plate portion.

FIGS. 16 (a) and (b) are explanatory diagrams to describe vertical movement of the top plate portion performed by the support mechanism, FIG. 16(a) illustrates a lowest position of the top plate portion, and FIG. 16(b) illustrating a highest position of the top plate portion.

FIGS. 17(a) and (b) are explanatory diagrams to describe front-back movement and swing movement of the top plate portion performed by the support mechanism, FIG. 16(a) illustrates a most front end position of the top plate portion, and FIG. 16 (b) illustrates a highest position when the top plate portion swings.

FIG. 18 is a diagram illustrating a relation between a movable range of the top plate portion and a cable storage unit.

FIG. 19 is a plan view illustrating an ultrasonic imaging device according to a different embodiment.

### DESCRIPTION OF EMBODIMENTS

In the following, embodiments of an ultrasonic imaging device and an ultrasonic imaging device cart will be described.

An ultrasonic imaging device 100 according to the present embodiment includes an instrument body 30 provided with a display unit 33 and an operation unit 31, a cart portion 10 supporting the instrument body 30, a top plate portion 20 supported by the cart portion 10 and mounted with the operation unit 31, and a probe 50 connected to the instrument body 30 via a cable 53. The top plate portion 20 includes a probe holder portion 22 located adjacent to a surface mounted with the operation unit 31 and configured to hold the probe, and the cart portion 10 is formed with a storage recess 45 continuous to a side surface from a front surface where an operator faces at the time of ultrasonic examination.

Further, an ultrasonic imaging device cart 200 according to the present embodiment includes a base seat 11 mounted with an ultrasonic imaging device (instrument body 30, etc.), a top plate portion 20 supported by the base seat 11 and configured to mount at least a part of the ultrasonic imaging device, a support mechanism 70 for the top plate portion 20, and a housing 40 to house the support mechanism 70. The base seat 11 includes a raised portion 13 surrounding an outer periphery of the housing 40 fixed to the base seat, and the storage recess 45 is formed between the raised portion of the base and the outer periphery of the housing.

The ultrasonic imaging device 100 and the cart 200 of the present embodiment further can have following features.

For example, the probe holder portions 22 are disposed on both sides of the top plate portion 20, and the storage recess 45 continues from the front surface to both side surfaces of the cart portion 10.

The cart portion 10 includes the base seat 11 provided with wheels 15, and the housing 40 fixed on the base seat 11 and including support mechanism 70 for the top plate portion 20. The base seat 11 includes the raised portion 13 surrounding the outer periphery of the housing 40 fixed to the base seat 11, and the storage recess 45 is formed as a space between the raised portion 13 of the base seat 11 and the outer periphery of the housing 40. The housing 40 includes a front surface panel 41 and two side surface panels 42, 43, and a rear surface panel 44, and at least one of the two side surface panels 42, 43 is formed with a recessed portion 42a at a position where the storage recess 45 is formed. Alternatively, a distance between the two side surface panels 42, 43 may be gradually narrowed toward the top plate portion 20 from a fixing portion with the base seat 11.

The storage recess 45 can include a lid portion 14 capable of changing capacity thereof. Alternatively, a removable lid portion can be provided.

The probe holder portion 22 includes a probe holding members 21 formed with a plurality of cutout portions into which the probes can be inserted, and an upper surface of the probe holding member is inclined relative to a horizontal surface. The inclination is, for example, an inclination along an arrayed direction of the plurality of cutout portions and/or an inclination directed to a side surface of the housing.

The probe holding member 21 can be provided with a jelly holder 23 in proximity to the plurality of cutout portions 22. The jelly holder 23 is disposed at a position most distant from a most front surface of the device relative to the plurality of cutout portions, and preferably, an upper surface of a portion provided with the jelly holder 23 of the probe holding member 21 is inclined most.

In the following, the ultrasonic imaging device of the present embodiment will be described with reference to the drawings. In the following description, a surface at which an operator faces the operation unit mounted on the top plate portion is referred to as a front surface of the device, the other side thereof is referred to as a rear surface, and surfaces on both sides of the front surface is referred to as side surfaces for convenience. Further, a direction orthogonal to the front surface is referred to as a front-rear direction, and a direction orthogonal to the side surface is referred to as a crosswise direction.

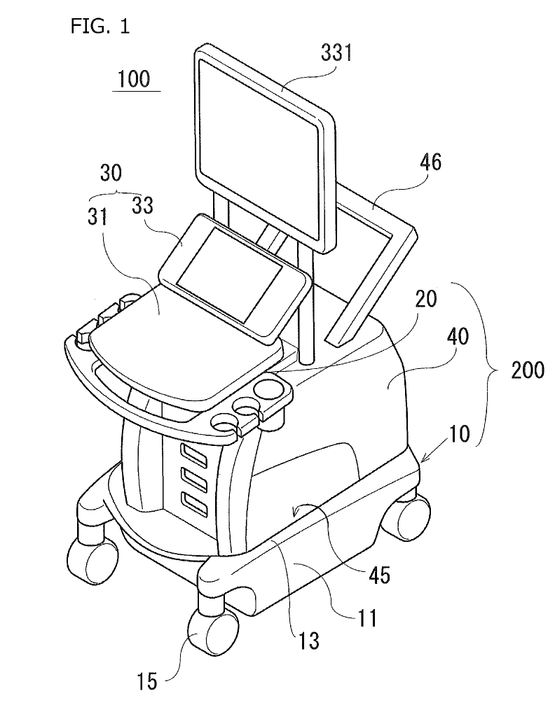

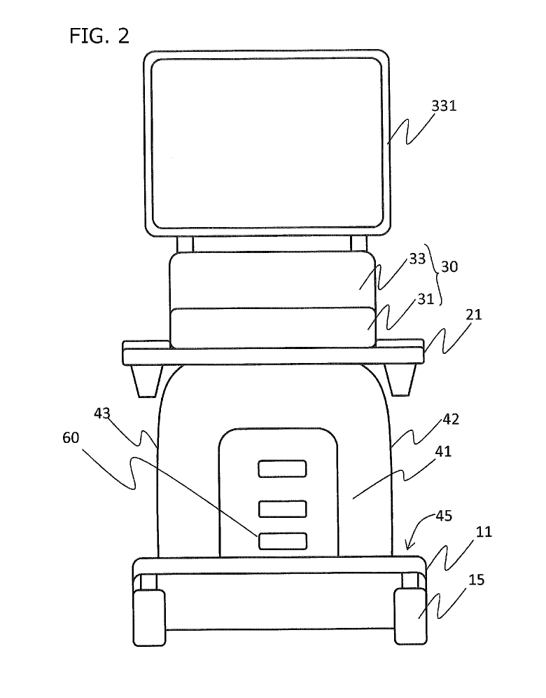

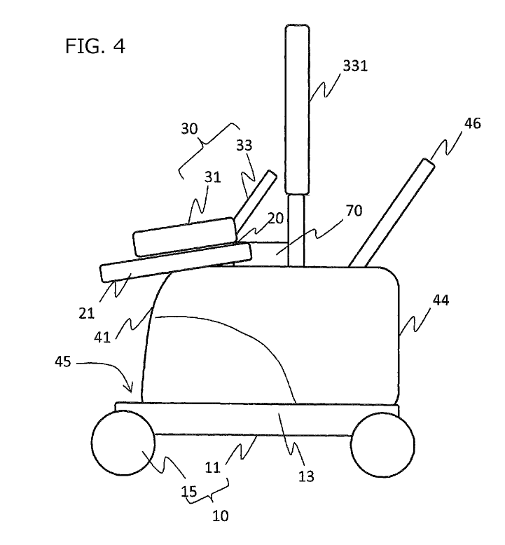

FIG. 1 is a perspective view illustrating an entire ultrasonic imaging device according to an embodiment and FIGS. 2 to 4 are a front view, a plan view, and a side view thereof. The ultrasonic imaging device includes the cart portion 10, the top plate portion 20 supported on the cart portion 10 by the support mechanism 70 (FIG. 4), the instrument body 30 mounted on the top plate portion 20, and the housing (cover portion) 40 to cover the support mechanism of the cart portion 10. Further, a display 331 is provided separately from the display unit disposed at the instrument body 30.

The cart portion 10 is formed of, as illustrated in FIGS. 1 and 2, the base seat 11 and the wheels 15 respectively fixed to a plurality of points of the base seat 11. The number of the wheels 15 is not limited, but according to the embodiment illustrated, four wheels capable of changing a traveling direction are provided. In the wheels, for example, a lock lever to lock/unlock rotation of the wheels can be provided.

The base seat 11 is formed with an edge portion (raised portion) 13 rising along the outer periphery thereof in a direction substantially perpendicular, and a space to be the storage recess 45 for a probe cable described later is formed between an inner surface of the raised portion 13 and an outer surface of the housing 40.

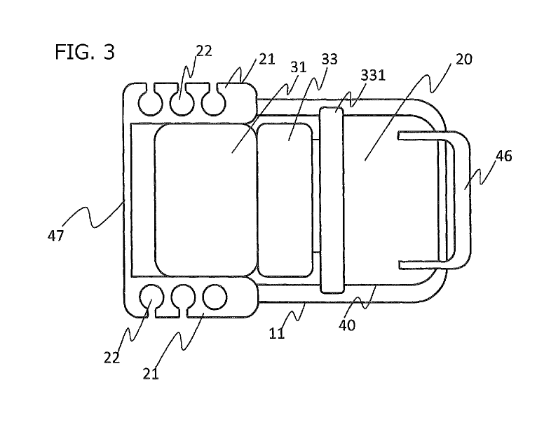

The top plate portion 20 is a table to mainly mount the operation unit 31 of the instrument body 30, and supported by the later-described support mechanism in a manner movable in the vertical direction and the front-rear direction with respect to the base seat 11 of the cart portion 10, and also in a pivotable manner within a range of a predetermined angle. On amounting surface of the top plate portion 20 where a bottom surface of the operation unit 31 contacts, the probe holding member 21 is integrally formed in manner projecting in the crosswise direction as illustrated in FIG. 3. Further, a handle 47 to operate a position of the top plate portion 20 is formed continuously with the probe holding member 21 in front of the mounting surface. The plurality of probe holder portions 22 is formed on the probe holding members 21 on both sides respectively. The probe holding member 21 and the probe holder portion 22 will be described in detail later.

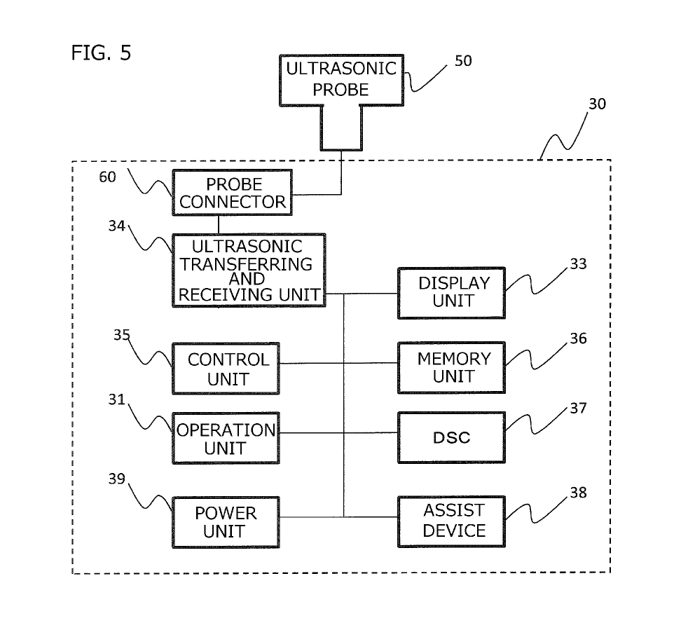

The instrument body 30 includes the operation unit 31 on which an operational button, a trackball, a keyboard, etc. for an operator to input a necessary condition and a command for examination are disposed, and the display unit 33 to display a GUI, an ultrasonic image, etc. necessary for operation, and further an electronic circuit needed to perform ultrasonic imaging is incorporated. A functional block diagram of the instrument body 30 is illustrated in FIG. 5.

The instrument body 30 is formed of a probe connector 60 to which the probe 50 is connected, an ultrasonic transferring and receiving unit 34, a control unit 35, a memory unit 36, digital scan converter (DSC) 37, an assist device 38, a power unit 39, etc. in addition to the above-described operation unit 31 and the display unit 33.

The ultrasonic transferring and receiving unit 34 transmits an electric signal to the probe 50 and also receives an electric signal from the probe 50 to perform phasing and so on. The DSC 37 scans a receiving signal on a time axis, and performs processing to display the signal as a two-dimensional image on a display panel of the display unit 33. The power unit 39 includes a terminal to supply power from a power source, such as a battery, and an external power source. The control unit 35 controls operation of the respective units. Further, necessary data to operate the respective units, image data being processed, a result thereof, etc. are stored in the memory unit 36.

Note that the configuration of the instrument body 30 illustrated in FIG. 5 is an example, and a configuration missing a part of the components or added with a different component may be also possible.

In the embodiment illustrated in FIGS. 1 to 4, the instrument body 30 is removed from the top plate portion 20, and functions as a notebook ultrasonic imaging device by itself. The operation unit 31 is integrally formed on the instrument body incorporated with the electronic circuit and the like, and the display unit (display panel) 33 is connected to the instrument body in a foldable manner. Note that a part of the electronic circuit needed to perform ultrasonic imaging can be disposed inside the housing 40 supporting the top plate portion 20 separately or duplicate with the instrument body 30.

The housing 40, namely, the cover for the support mechanism supporting the top plate portion 20 has an approximately rectangular parallelepiped shape surrounded by the front surface panel 41, side surface panels 42, 43, and rear surface panel 44, and has a dimension of an outer periphery at a lower portion fixed to the base seat 11 smaller than a dimension of an inner periphery of the raised portion 13 of the base seat 11. The recessed portion 45 is formed continuously from both sides to the front surface between the raised portion 13 and the housing 40. The recessed portion 45 functions as the storage recess 45 to house a cable of the probe 50 although described later in detail.

The front surface panel 41 of the housing 40 is inclined rearward from the lower portion to an upper portion thereof in order to secure a movable area of the top plate portion 20 as illustrated in FIG. 4. Further, a recessed portion is formed at a center portion of the front surface panel 41, and the probe connector 60 (FIG. 2) is formed in the recessed portion. The probe connector 60 is electrically connected to the instrument body 30 mounted on the top plate portion 20 via a cable not illustrated. The both side surface panels 42, 43 and the rear surface panel 44 of the housing 40 stand almost perpendicular from the lower portion, and the side surface panels 42, 43 are gently inclined inward at a portion in a portion close to the top plate portion 20, and a distance between the side surface panels is gradually narrowed.

An operation handle 46 to operate travel of the ultrasonic imaging device provided with the cart is disposed at the upper portion of the housing 40. Further, a handle or the like to operate vertical movement and rotation of the top plate portion 20 is preferably disposed on the front surface side of the cart portion 10. According to the embodiment illustrated, the handle 47 to operate rotation of the top plate portion 20 is formed on the front surface side, continuously from the probe holding members 21 on both sides. The vertical operation can be executed by a pedal or a lever.

According to the embodiment illustrated, the operation handle 46 is integrally provided with the housing at the upper portion of the housing 40 such that a gripping portion is positioned at a rear portion of the housing 40. The operator stands on the rear side of the device and can move the device forward by pushing the operation handle 46. Further, a vertical position of the top plate portion 20 fixed to the support mechanism 70 can be adjusted by operating the handle, a pedal, or the like not illustrated. Meanwhile, the handle or the pedal may include a lock handle (not illustrated) to lock the position of the top plate portion 20, by which the top plate portion 20 can be fixed at an optional vertical position.

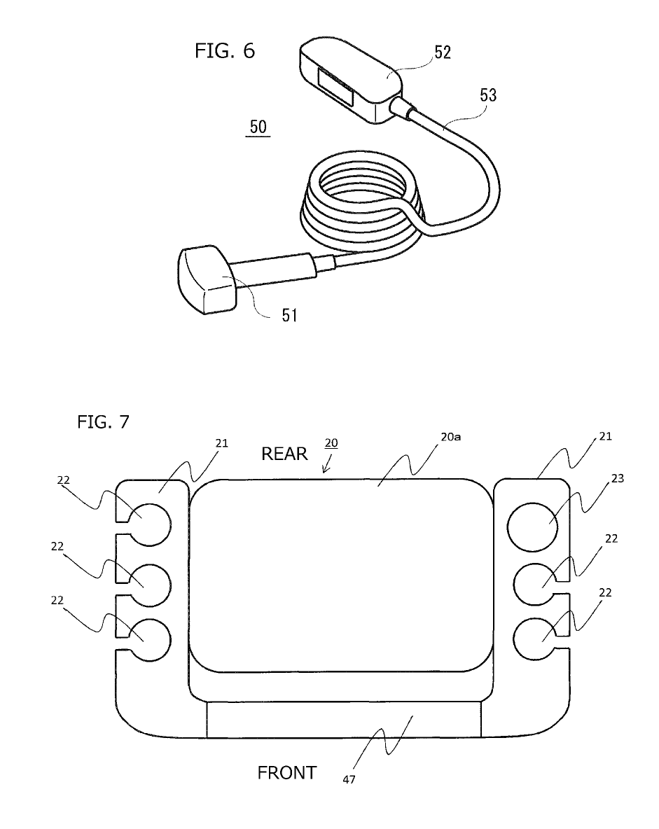

The probe 50 transmits an ultrasonic signal upon receipt of an electric signal from the ultrasonic transferring and receiving unit 34 of the instrument body 30, and receives an echo signal of ultrasonic reflected inside an examination target, and then convert the echo signal to an electric signal. As illustrated in FIG. 6, the probe 50 includes a probe body 51 provided with a piezoelectric element array in which piezoelectric elements are arrayed in one-dimensional direction or two-dimensional directions, a connector 52 connected to the probe connector 60, and a cable 53 connecting the probe body to the connector. The probe illustrated in FIG. 6 is an example, and there may be various kinds of probes having probe bodies 51 of different shapes and sizes. A length of the cable 53 is not determined but is required to be a length equal to or longer than a distance from a general installation place of the device to an examination target, i.e., about 2.5 to 3 m.

Based on the above-described outline of the device, the respective units having features of the present embodiment will be described in detail.

First, the top plate portion 20, particularly, a structure of the probe holding member 21 provided with the probe holder portion 22 will be described with reference to FIGS. 7 to 10. FIGS. 7 to 9 are a plan view, a side view, and a front view of the probe holder portion 21 respectively. FIG. 10 is a perspective view illustrating a probe cup constituting the probe holder portion.

As illustrated in FIG. 7, the probe holding members 21 are disposed continuously from a mounting surface 20a for the instrument body 30 on the top plate portion 20 on right and left sides thereof. The probe holder portion 22 includes the circular cutout portions formed on these probe holding member 21, and a probe cup 25 (FIG. 10) to be fitted into the cutout portion. In the example illustrated, three cutout portions are formed at one of the right and left probe holding members 21 (left side in the drawing), and all of the cutout portions are the cutout portions to be the probe holder portions 22. The two cutout portions to be the probe holder portions 22 and a circular cutout portion (hole) to be the jelly holder 23 are formed at the probe holding member 21 on the other side (right side in the drawing).

The structures of the probe holder portions 22 are the same except for the jelly holder 23. Therefore, a description will be given for one probe holder portion. The cutout portion has a shape in which a part of a circle is opened to the outside, and the probe cup 25 to hold the probe is fitted into the circle portion. The probe cup 25 is a cylindrical cup having openings on both sides as illustrated in FIG. 10, and a flange 25a is formed on one end, and a slit 25b is formed from one end to the other end. The probe cup is set to the cutout portion such that the slit 25b matches the opened portion of the cutout portion while the flange 25a is positioned on the upper side. The probe cable 53 can be put in and out from the slit of the probe cup 25, thereby achieving to easily store, in the probe holder portion 22, the probe 50 being connected to the cable 53, and also achieving to eject the same.

The jelly holder 23 is used to place a container containing jelly to be applied to the examination target at the time of ultrasonic examination. The jelly container is generally formed of a bottle having a height (length) higher than the probe body 51, and a cup 27 deeper than the probe cup is fitted into the cutout portion for the jelly holder in accordance with the height. The opened portion or the slit for the probe cable are not needed in the cutout portion for jelly and the cup 27 for jelly.

The probe holder portion 22 and the jelly holder 23 are disposed aligned along the side surface. Preferably, the jelly holder 23 is disposed on the rear side of the probe holder portion 22.

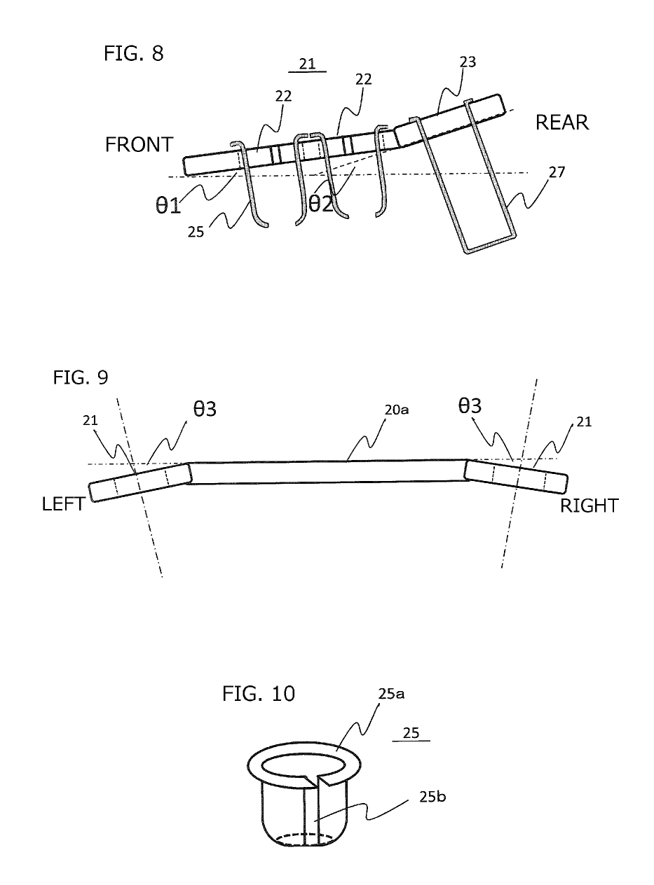

The top plate portion 20 of the present embodiment is characterized in that the upper surface of the probe holding member 21 is inclined relative to the horizontal surface. The inclination may be possible in any of the front-rear direction and the crosswise direction. In FIG. 8, the inclination is made in the front-rear direction, namely, an arrayed direction of the probe holder portion 22 and the jelly holder 23. The inclination in the front-rear direction is formed such that a height of the probe holder portion 22 from a floor surface becomes gradually higher from the front side to the rear side. An inclination angle θ1 is not limited, but approximately 1° to 30°. An inclined surface may not form a constant angle relative to the horizontal surface, and the angle may be changed. For example, the inclination angles θ1 and θ2 may be set different between a surface on which the probe holder portion 22 is disposed and a surface on which the jelly holder is disposed. Alternatively, the inclination angle may continuously or gradually become larger from the front side to the rear side.

In accordance with such an inclination of the upper surface of the probe holding member 21, an axial direction of the probe cup is also inclined to front side with respect to the vertical direction. With this structure, at the time of putting in and out the probe from the probe holder portion 22, the operator facing the device just lift the probe obliquely to the front side and puts the same down obliquely to the rear side, instead of lifting up the probe straight above and putting down the same from straight above. The closer to the operator the probe holder portion 22 is located, the lower the same is positioned. Therefore, the probe holder even positioned distant from the operator can be easily handled. Further, since the cable of the probe is oriented rearward from the vertical direction, the cable can be easily stored in the cable storage recess 45 described later in detail.

Further, the jelly container generally has the height (length) higher than the probe body. Therefore, operability at the time of putting in and out the probe can be more improved by making the inclination of the surface provided with the jelly holder 23 larger than the surface provided with the probe holder portion 22.

FIG. 9 illustrates the inclination of the upper surface of the probe holding member 21 in the crosswise direction with respect to the horizontal surface. More specifically, the probe holding members 21 located on the right and left sides of the top plate portion 20 respectively have inclinations formed such that the height from the floor surface becomes slightly lower toward an end portion side from the connecting portion with the surface 20a of the top plate portion 20. In the case of forming such inclinations, the axial direction of the probe cup is also inclined from the vertical direction as indicated by a dot-and-dash line, and the lower end thereof is positioned on a more inner side (the housing 40 side) than the upper end thereof. Therefore, the probe cable stored in the probe holder portion 22 is more easily stored in the later-described cable storage recess 45 along the housing 40. An angle θ3 of an inclination in the crosswise direction is not limited, but approximately 1° to 30°.

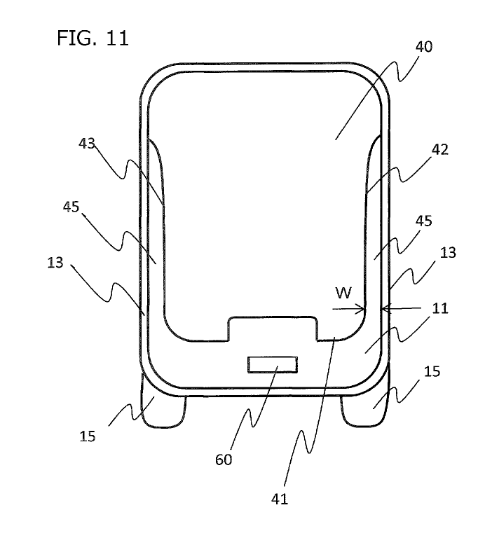

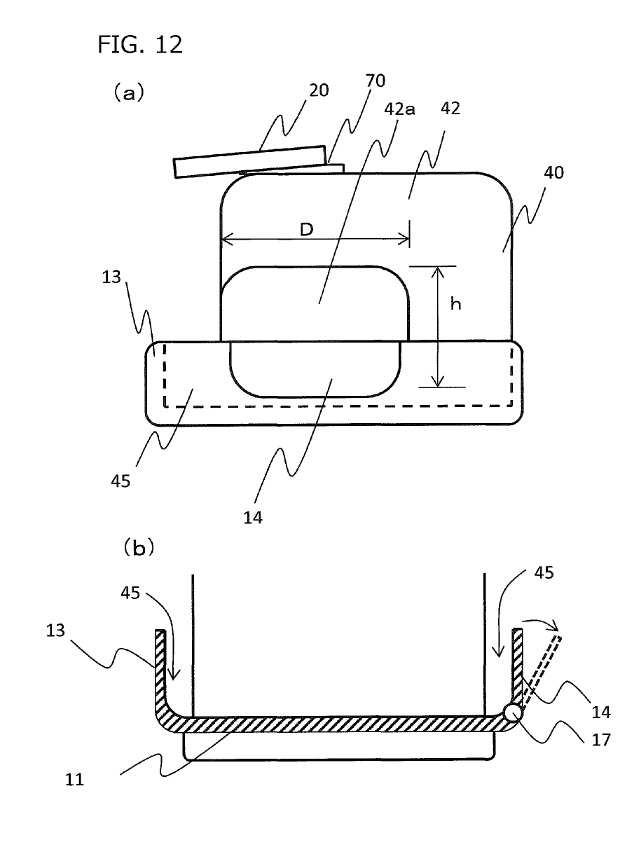

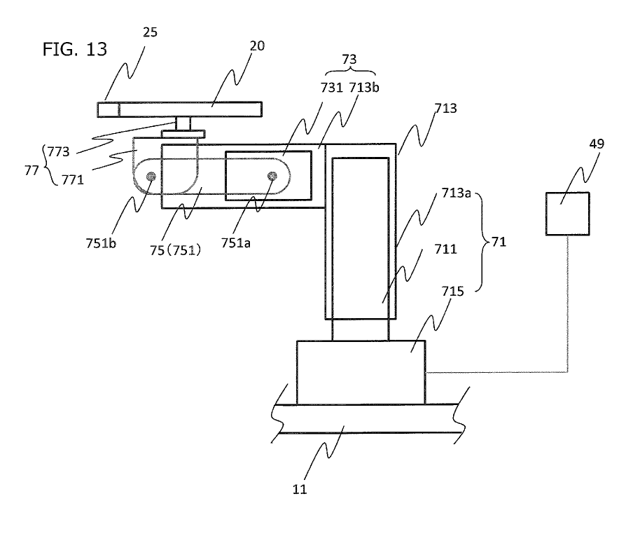

Next, the probe cable storage recess 45 formed at the cart portion 10, particularly, between the base seat 11 and the housing 40 will be described with reference to FIGS. 11 and 12. FIG. 11 is a plan view of the base seat 11, FIG. 12(a) is a partial side view, and FIG. 12(b) is a partial cross-sectional view, in which the inside of the housing 40 and a portion above the housing 40 of the ultrasonic imaging device of the present embodiment are partially omitted.

As illustrated in FIG. 11, the base seat 11 is formed as a recessed portion in which an upper surface is substantially opened by the raised portion 13 vertically disposed along the outer periphery thereof, and the housing 40 is fixed so as to cover a bottom of the recessed portion. Spaces are formed between the side surface panels 42, 43 of the housing 40 and side surfaces of the raised portion 13 and between the front surface panel 41 of the housing 40 and a front surface of the raised portion 13 respectively, and these spaces form the probe cable storage recess 45 continuously. A size of the space on the side surface sides is not particularly limited, but designed to have the size that can allow the cable to move in the case where the cable moves due to movement and pivot of the top plate portion 20 described later. According to the embodiment illustrated, a length in the front-rear direction is smaller than a width (dimension in the front-rear direction) of the side surface of the base seat 11, and larger than a half thereof. Further, preferably, a width W in the crosswise direction is about two to five times a cable diameter.

Further, a side surface cover (lid portion) 14 may be attached to the side surface of the raised portion 13 as illustrated in FIGS. 12(a) and 12(b). The side surface cover 14 can have a structure openable and capable of adjusting an opening angle by connecting, for example, one end portion to the raised portion 13 or the base seat 11 via a hinge 17. By adopting such a structure, the width W in the crosswise direction of the above-described storage recess 45, namely, capacity of the storage recess 45 can be adjusted with the opening angle of the side surface cover 14.

Further, as a different example, the side surface cover 14 can be formed as a removable structure. In this case, by preparing a plurality of side surface covers 14 having different shapes, the capacity of the storage recess 45 can be adjusted same as the embodiment illustrated in FIG. 12.

Since the capacity of the storage recess 45 is made adjustable, flexible handling can be performed even in the case where the probe has a different cable length, or the number of the probes housed in the probe holder portion 22 is changed. Further, in the case where the number of the probes, namely, the number of the cables is plural, a partition plate (not illustrated) can be inserted parallel to the side surface panel.

Preferably, the space on a front surface side of the storage recess 45 is continuous to the space on the side surface side, and further has a width (front-rear direction) substantially same as the width W on the side surface side. According to the embodiment illustrated in FIG. 11, a probe connector 60 is formed on the floor portion in the space on the front surface side of the storage recess 45, and can be used same as the probe connector 60 formed on the front surface 41 of the housing 40 illustrated in FIG. 2. According to the ultrasonic imaging device of the present embodiment, the space having sufficient capacity is provided as the probe cable storage recess continuous from the front surface to the side surfaces. Therefore, the plurality of probes can be kept connected to the plurality of probe connectors 60. Therefore, to increase the probe connectors 60 by utilizing the floor portion of the storage recess 45 is meaningful.

A description will be given for the shapes of the side surface panels 42, 43 of the housing 40 to improve storage capacity of the storage recess 45. Since the shapes of the side surface panels 42, 43 are bilaterally symmetric and the same, the side surface panel 42 on the right side will be described as a representative. The side surface panel 42 of the housing 40 is formed of one or plurality of plate material having a flat shape or a gentle curved shape, and forms the side surface of the housing 40. Further, as illustrated in FIG. 12(a), a recessed portion 42a recessed inward is formed corresponding to the storage recess 45 positioned on the side surface side. A width (dimension in the front-rear direction) D of the recessed portion 42a corresponds to a length in the front-rear direction of the storage recess 45, and a height h is not particularly limited but approximately a half of a height of the side surface panel. A depth of the recessed portion 42a may be almost same as the width of the storage recess 45 or less than that. The recessed portion 42a of the side surface panel 42 is connected to a right end of the front surface panel 41 with a curved panel.

By forming the above-described recessed portion 42a on the side surface panel 42, the probe cable stored at the probe holder portion 22 above the recessed portion 42a can be stored in the recessed portion 42a and the storage recess 45 along the side surface panel 42 without largely moving outward from the side surface. Further, the recessed portion 42a can also function as a storage unit for a document such as a chart, for example.

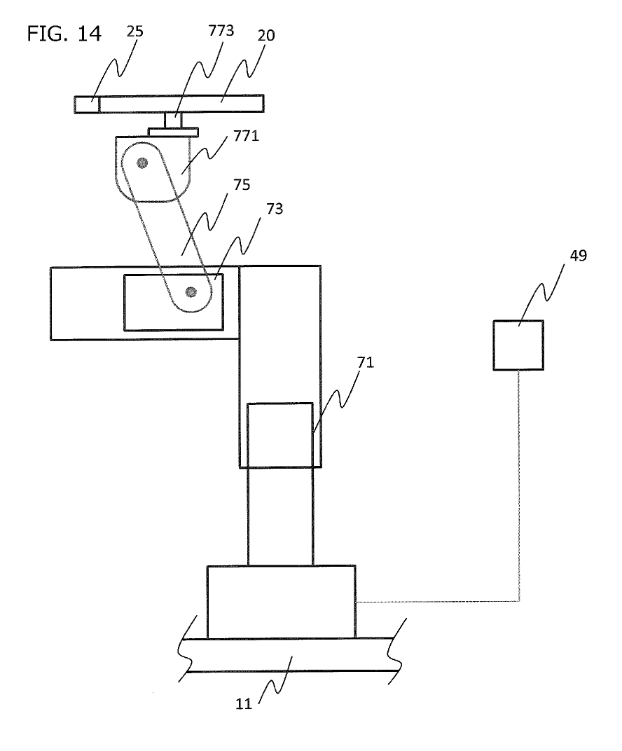

Next, the support mechanism for the top plate portion 20 will be described with reference to FIGS. 13 and 14. In FIGS. 13 and 14, the support mechanism connecting the top plate portion 20 to the base seat 11 is schematically illustrated, and other members are omitted.

The support mechanism 70 for the top plate portion 20 is roughly divided into a vertical slide mechanism 71, a horizontal slide mechanism 73, a swing mechanism 75, and a rotation mechanism 77.

The vertical slide mechanism 71 includes a pole 711, a vertical slider 713 fitted with the pole 711 and movable in the vertical direction, and a drive mechanism 715 of hydraulic cylinder or the like to drive the vertical slider 713. Further, a mechanism to move the pole 711 in the front-rear direction relative to the base seat 11 may be provided as well. The drive mechanism 715 is operated by an operation tool 49 such as an operation handle or a foot pedal provided on the front side of the housing. The vertical slider 713 includes a vertical portion 713a and a horizontal portion 713b, and the vertical portion 713a is fitted to the pole 711 and moved in the vertical direction together with the horizontal portion 713b in accordance with operation of the operation tool 49.

The horizontal slide mechanism 73 is fitted to the horizontal portion 713b of the vertical slider 713, and includes a horizontal slider 731 movable in the horizontal direction. One end 751a of a swing arm 751 constituting the swing mechanism 75 is rotatably fixed to one end of the horizontal slider 731. The rotation mechanism 77 is fixed to the other end 751b of the swing arm 751. The swing arm 751 can swing around the one end 751a fixed to the horizontal slider 731 in a range from a position where the swing arm is parallel to the horizontal slider 731 illustrated in FIG. 13 to a position where the swing arm becomes substantially vertical as illustrated in FIG. 14.

The other end 751b of the swing arm 751 is rotatably fixed to the rotation mechanism 77, and the rotation mechanism 77 includes a fixing portion 771 having a horizontal fixed surface, and a rotary shaft 773 fixed to the fixed surface of the fixing portion 771 and rotatably supporting the top plate portion 20, and a stopper (not illustrated) to restrict a rotation amount of the top plate portion 20 supported by the rotary shaft 773.

Movement and rotation of the top plate portion 20 by the horizontal slide mechanism 73, swing mechanism 75, and rotation mechanism 77 are executed by the operator operating the operation handle 47 provided at the top plate portion 20.

Since known mechanisms can be adopted for the slide mechanism and the rotation mechanism, a detailed description therefor will be omitted.

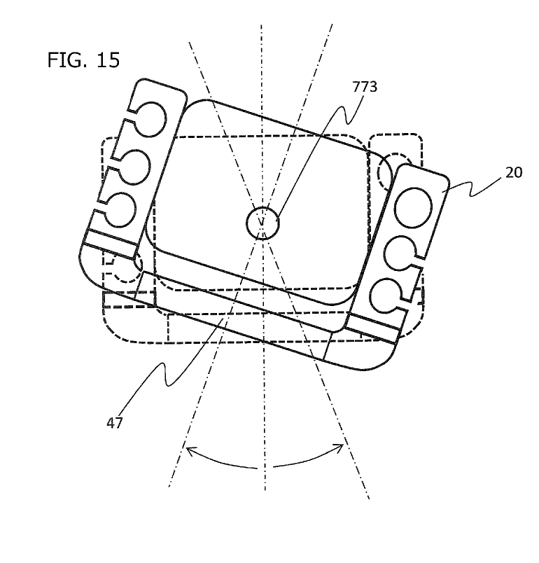

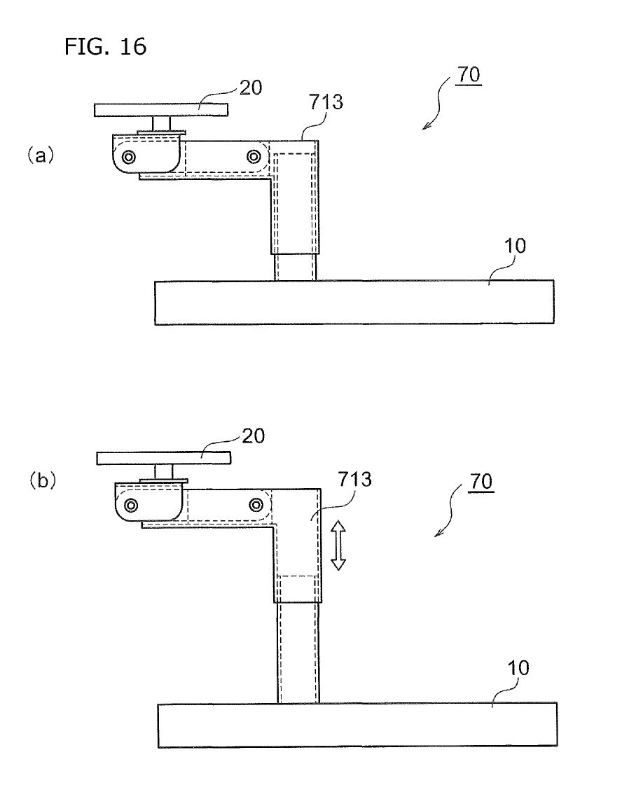

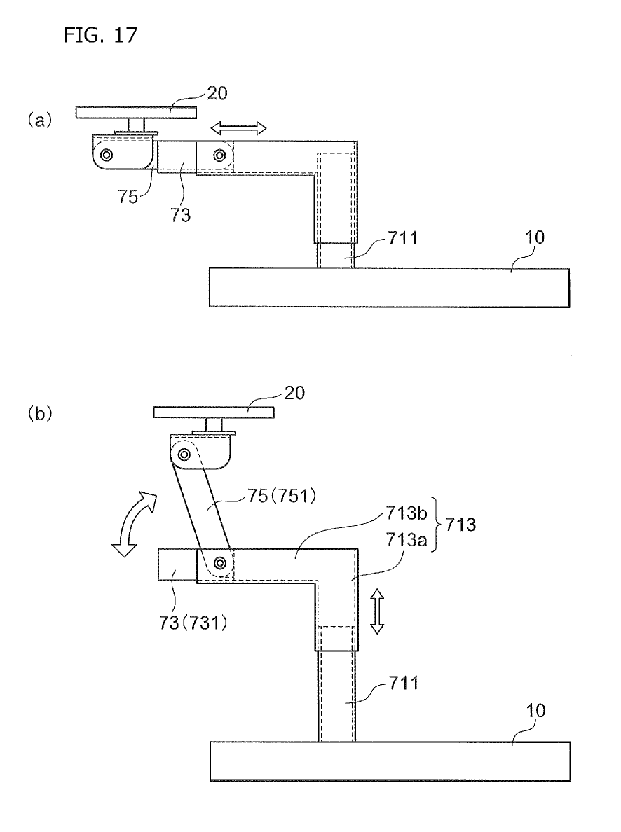

The movable range of the top plate portion 20 supported by the above-described support mechanism 70 will be described with reference to FIGS. 15 to 17(b). FIG. 15 is a top view of the top plate portion 20, and FIGS. 16(a) and 16(b) are explanatory diagrams to describe vertical movement of the top plate portion by the vertical slide mechanism 71, and FIGS. 17(a) and 17(b) are explanatory diagrams to describe movement of the top plate portion by the horizontal slide mechanism 73 and swing mechanism 75.

In FIG. 15, an initial position (reference position) of the top plate portion 20 is indicated by a chain line, and pivot positions are indicated by a solid line. Dot-and-dash lines indicate the movable range of the top plate portion 20 in a rotational (pivot) direction. As illustrated, the top plate portion 20 is rotated around the rotary shaft 773 by the above-described rotation mechanism, and can pivot in the crosswise direction up to a predetermined angle restricted by the stopper from the initial position (chain line) where a center of the handle 47 coincides with a center of the front surface panel of the housing 40. The pivot angle is not particularly limited, but about ±25°, for example.

FIGS. 16(a) and 16(b) are the diagrams illustrating the movable range of the top plate portion 20 by the vertical slider 713, FIG. 16(a) illustrates a state in which the top plate portion 20 is located at a lowermost end, and FIG. 16(b) illustrates a state in which the top plate portion 20 is located at an uppermost end. The vertical slider 713 can move within the movable range from the lowermost end to the uppermost end. In the positions illustrated in both FIGS. 16 (a) and 16 (b), the horizontal slider 731 is positioned most rearward, and the swing arm 751 is located at a position parallel to the horizontal slider 731.

Movement of the top plate portion 20 by using the horizontal slide mechanism 73 and swing mechanism 75 can be performed at any position within the movable range in the vertical direction of the vertical mechanism 71, and the height of the top plate portion 20 can be adjusted within a swing range of the swing arm 751 by operating the operation handle 47 of the top plate portion 20 to move the swing mechanism 75 in a state that the vertical mechanism 71 is fixed by the lock handle. Further, by moving one or both of the horizontal slide mechanism 73 and the swing mechanism 75, the top plate portion 20 can be set at a desired position within the movable range of the horizontal slide mechanism 73 in addition to the movable range in the front-rear direction by the swing mechanism 75.

FIG. 17(a) illustrates a state in which the top plate portion 20 is pulled most frontward by the horizontal slide mechanism 73 at the lowermost position in the vertical direction illustrated in FIG. 16(a), and FIG. 17(b) illustrates a state in which the top plate portion 20 is moved to the highest position by the swing mechanism 75 in the uppermost position in the vertical direction illustrated in FIG. 16(b). The position illustrated in FIG. 17(b) is the position most rearward within the movable range in the front-rear direction by the swing mechanism 75.

The movable range of the top plate portion 20 is not limited, but as an example, the vertical movable range (stroke) by the vertical slide mechanism is 120 mm, the horizontal movable range (stroke) by the horizontal slide mechanism is 150 mm, the swing range of the swing arm 751 by the swing mechanism 75 is from horizontal to 73 degrees, and a vertical stroke and a front-rear stroke brought by the swing is 180 mm.

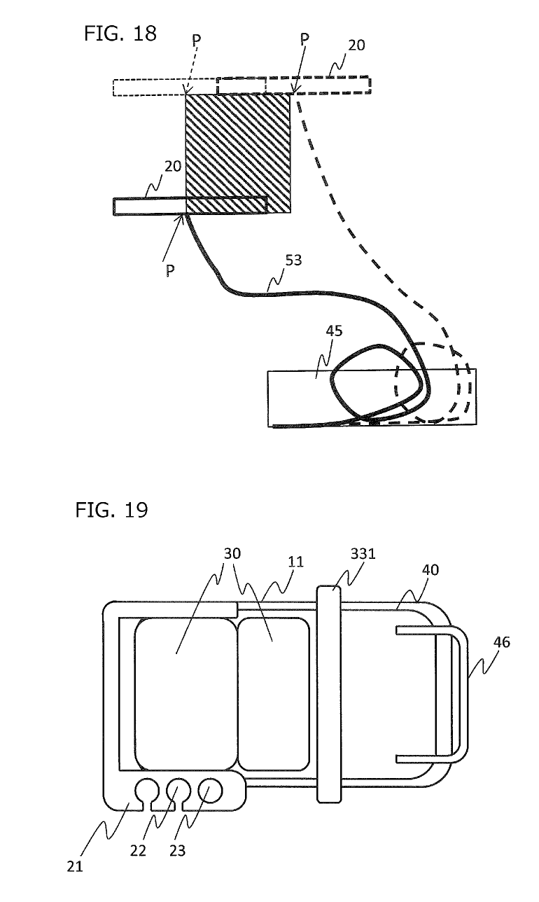

In the case where the probe 50 is stored at the probe holder portion 22 of the top plate portion 20 which moves within the above-described movable range, a relation between the probe 50 and the cable storage unit 45 is schematically illustrated in FIG. 18. In the drawing, a shaded area is the movable area of a point P of the top plate portion 20 (e.g., probe holder portion located at a center) in the vertical and crosswise directions, and a storage state of the cable 53 in the case where the top plate portion 20 is located at the position illustrated in FIG. 17(a) is indicated by a solid line and a storage state of the cable 53 in the case where the plate top plate portion 20 is located at the position illustrated in FIG. 17 (b) is indicated by a chain line. As illustrated, the probe cable storage recess 45 is formed continuously from the front surface to the side surface of the housing 40. Therefore, even when the connecting portion between the probe body 51 stored at the probe holder portion 22 and the cable 53 is moved by movement of the top plate portion 20, excessive force is not applied to the cable 53 having one end connected to the probe connector 60 on the front surface of the housing 40, and also the cable 53 is kept stored inside the storage recess 45 without coming outward from the side surface of the housing 40.

Further, FIG. 18 illustrates the case where the top plate portion 20 is in the initial position of pivot, but in the case where the top plate portion 20 pivots as illustrated in FIG. 15, the position of the point P is changed in a direction orthogonal to the drawing paper of FIG. 18. For example, in the case where the top plate portion 20 pivots from a front side to a back side of the drawing paper, the cable 53 is also pulled along with the pivoting, but looseness of the cable 53 can resolve the moved amount of pulling. Further, since the storage recess 45 is continuously formed up to the front surface, the surplus loose portion can be stored in the storage recess 45 of the front surface. In contrast, in the case where the top plate portion 20 is moved from the back side to the front side, looseness of the cable 53 is increased. But, since the recessed portion 42a is formed on the side surface panel 42 of the housing 40, the cable is prevented from coming outward from the side surface of the housing 40.

Meanwhile, in FIG. 18, only one probe cable is illustrated, but the case is the same in the case where a plurality of probes is housed. Especially, entangling of the cables can be surely prevented by inserting a partition plate parallel to the side surface panel 42(43) of the housing 40.

In the above, an embodiment of the ultrasonic imaging device according to the present invention has been described using the drawings, but the ultrasonic imaging device illustrated in the drawings is just an example and various kinds of modifications can be made. For example, while the embodiment including probe holding members 21 on both sides of the top plate portion 20 is illustrated in FIGS. 1 to 3, etc., but the probe holding member 21 can be disposed only one side of the top plate portion 20 as illustrated in FIG. 19. In this case, the cable storage recess 45 is to be formed continuously from the front surface to the side surface corresponding to a side where the probe holding member 21 is provided, and the storage recess 45 is not necessarily formed on the side where the probe holding member 21 is not provided.

Further, according to the above-described embodiment, the example in which the support mechanism for the top plate portion 20 is formed of the three mechanisms including the vertical slide mechanism, horizontal slide mechanism, and swing mechanism has been described, but these mechanisms are not essential and a part or all thereof may not be necessarily provided. In other words, the present invention can be applied to an ultrasonic imaging device in which the top plate portion 20 is fixed to the cart portion 10 by a pole or the like, for example.

Further, according to the above-described embodiment, the notebook instrument which is capable of opening and closing the operation unit 31 and the display unit 33 and can be removable from the top plate portion 20 has been described as the instrument body 30, but the instrument body can take various forms and may also be integrally formed with the top plate portion 20.

The shapes and positions of the respective members illustrated in other drawings may be changed in various ways.

### INDUSTRIAL APPLICABILITY

According to the present invention, a mobile ultrasonic imaging device having excellent operability for a probe is provided. This improves work performance of ultrasonic examination even in a place having space restriction.

### REFERENCE SIGNS LIST

10 cart portion
20 top plate portion
21 probe holding member
22 probe holder portion
23 jelly holder
30 instrument body
31 operation unit
33 display unit
40 housing
41 front surface panel
42, 43 side surface panel
45 storage recess
46, 47 operation handle
50 probe
51 probe body
52 connector
53 cable
60 probe connector
70 support mechanism
100 ultrasonic imaging device
200 ultrasonic imaging device cart

1. An ultrasonic imaging device, comprising:
an instrument body including a display unit and an operation unit;
a cart portion supporting the instrument body;
a top plate portion supported by the cart portion and mounted with the operation unit; and
a probe to be connected to the instrument body via a cable,
wherein the top plate portion includes a probe holder portion located adjacent to a surface mounted with the operation unit and configured to hold the probe, and the cart portion is formed with a storage recess continuous to a side surface from a front surface.

2. The ultrasonic imaging device according to claim 1, wherein the probe holder portions are provided on both sides of the top plate portion, and the storage recess is continuously formed from the front surface to the both side surfaces of the cart portion.

3. The ultrasonic imaging device according to claim 1, wherein
the cart portion includes a base seat provided with wheels, and a housing fixed onto the base seat and including a support mechanism for the top plate portion,
the base seat includes a raised portion surrounding an outer periphery of the housing fixed to the base seat, and
the storage recess is formed as a space between the raised portion of the base seat and the outer periphery of the housing.

4. The ultrasonic imaging device according to claim 3, wherein the housing includes two side surface panels, at least one of the two side surface panels is formed with a recessed portion at a place where the storage recess is formed, and the recessed portion is recessed inward of the housing.

5. The ultrasonic imaging device according to claim 3, wherein the housing includes two side surface panels, and a distance between the two side surface panels is gradually narrowed toward the top plate portion from a fixing portion with the base seat.

6. The ultrasonic imaging device according to claim 1, wherein the storage recess includes a lid portion capable of changing capacity of the storage recess.

7. The ultrasonic imaging device according to claim 1, wherein the storage recess includes a removable lid portion.

8. The ultrasonic imaging device according to claim 1, wherein the probe holder portion is formed of a probe holding member formed with a plurality of cutout portions into which the probe can be inserted, and an upper surface of the probe holding member is inclined relative to a horizontal surface.

9. The ultrasonic imaging device according to claim 8, wherein an upper surface of the probe holding member is inclined along an arrayed direction of the plurality of cutout portions and/or along a crosswise direction of the cart portion.

10. The ultrasonic imaging device according to claim 8, wherein the probe holding member includes a jelly holder in proximity to the plurality of cutout portions.

11. The ultrasonic imaging device according to claim 10, wherein the jelly holder is disposed at a position most distant from the front surface of the device relative to the plurality of cutout portions, and an upper surface where the jelly holder of the probe holding member is inclined most.

12. An ultrasonic imaging device cart, comprising:
a base seat configured to mount an ultrasonic imaging device;
a top plate portion supported by the base seat and configured to mount at least a part of the ultrasonic imaging device;
a support mechanism for the top plate portion, and
a housing configured to house the support mechanism,
wherein the base seat includes a raised portion surrounding an outer periphery of the housing fixed to the base seat, and a storage recess is formed between the raised portion and the outer periphery of the housing.
